(19) United States
(12) Patent Application Publication
(10) Pub. No.: US 2009/0112121 A1
(43) Pub. Date: Apr. 30, 2009

(54) BLEEDING APPARATUS

(76) Inventors: Min-Chieh Chuang, Tainan City (TW); Herbert Ho, Taipei City (TW)

Correspondence Address: NORTH AMERICA INTELLECTUAL PROPERTY CORPORATION, P.O. BOX 506, MERRIFIELD, VA, 22116, US

(21) Appl. No.: 12/125,020

(22) Filed: May 21, 2008

(30) Foreign Application Priority Data
Oct. 26, 2007 (TW) 096140385

(51) Int. Cl. A61B 5/15

(52) U.S. Cl. 600/573

(57) ABSTRACT

A bleeding apparatus includes a first stimulating component for pressing an examinee at a first simulating point so as to induce sense perception, and a second stimulating component for pressing the examinee at a second simulating point so as to induce sense perception. A distance between the first stimulating component and the second stimulating component is less than or equal to a two-point sense threshold of a human perception system. The bleeding apparatus further includes a bleeding component disposed between the first stimulating component and the second stimulating component for bleeding the examinee between the first stimulating point and the second stimulating point when the first stimulating component is pressing the examinee at the first simulating point and the second stimulating component is pressing the examinee at the second simulating point.

### BACKGROUND OF THE INVENTION

1. Field of the Invention

The present invention relates to a bleeding apparatus, and more particularly, to a bleeding apparatus capable of reducing pain of an examinee when bleeding.

2. Description of the Prior Art

Recently patients can use small examining instruments to examine themselves due to the progress of medical technology. For example, a diabetic can examine blood sugar by himself at home with a bleeding needle for collecting blood. Because the bleeding needle pricks to subcutaneous nociceptors of his skin, the examinee has obvious sensation of pain. It causes an obstacle of self-examination especially for chronic patients who need to use bleeding needles often. The patients repel bleeding due to sensation of pain causing health deterioration indirectly.

FIG. 1 illustrates a conventional bleeding apparatus 10. The bleeding apparatus 10 includes a rod 12. A contacting surface 14 is disposed on one end of the rod 12. An opening 16 is disposed on the contacting surface 14 so that a needle 18 installed inside the rod 12 is capable of protruding therethrough. The contacting surface 14 of the bleeding apparatus 10 contacts with skin of a user when bleeding. The needle 18 is capable of protruding through the opening 16 when turning on a switch (not shown in FIG. 1) on the rod 12. However, the user has obvious sensation of pain when the needle 18 pricks skin. There is a need to design a bleeding apparatus capable of reducing an examinee's pain when bleeding.

### SUMMARY OF THE INVENTION

It is therefore an objective of the present invention to provide a bleeding apparatus capable of reducing pain of an examinee when bleeding the examinee.

According to the present invention, a bleeding apparatus includes a first stimulating component for pressing an examinee's skin at a first simulating point so as to induce sense perception, and a second stimulating component for pressing the examinee's skin at a second simulating point so as to induce sense perception. A distance between the first and second stimulating components is less than or equal to a two-point sense threshold of a human perception system. The bleeding apparatus further includes a bleeding component disposed between the first and second stimulating components for bleeding the examinee when the first and second stimulating components are pressing the examinee at the first and second simulating points, respectively.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

The present invention will become more fully understood from the detailed description given herein below and the accompanying drawings which are given by way of illustration only, and thus are not limitative of the present invention, and wherein:

FIG. 1 illustrates a schematic diagram of a conventional bleeding apparatus;

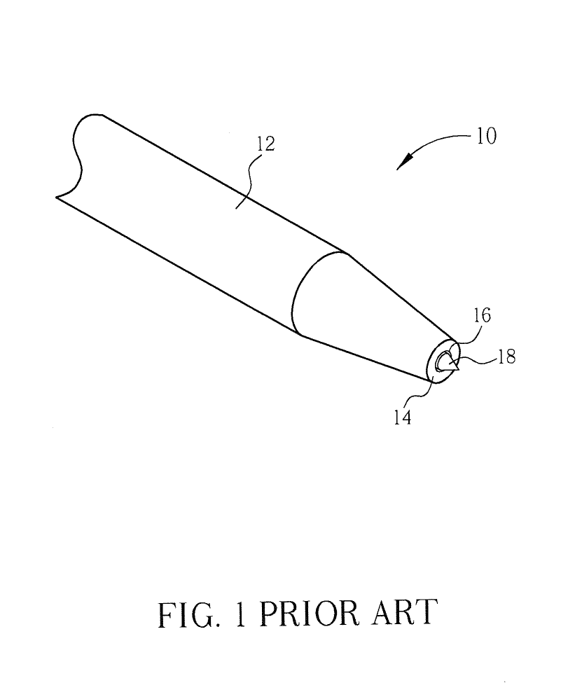

FIG. 2 illustrates a schematic diagram of a bleeding apparatus according to a first embodiment of the present invention;

FIG. 3 illustrates a schematic diagram of the bleeding apparatus of FIG. 2 bleeding an examinee;

FIG. 4 illustrates an end view of a bleeding apparatus according to a second embodiment of the present invention;

FIG. 5 illustrates an end view of a bleeding apparatus according to a third embodiment of the present invention; and

FIG. 6 illustrates an end view of a bleeding apparatus according to a fourth embodiment of the present invention.

### DETAILED DESCRIPTION

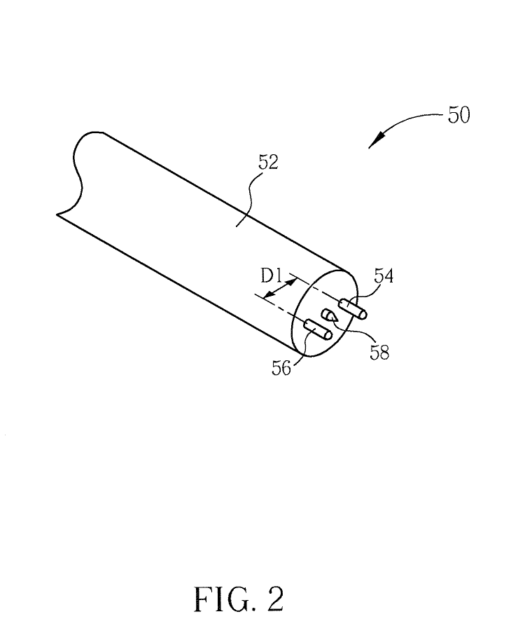

FIG. 2 illustrates a schematic diagram of a bleeding apparatus 50 according to a first embodiment of the present invention. As shown in FIG. 2, the bleeding apparatus 50 includes a rod-type structure 52, a first stimulating component 54, a second stimulating component 56, and a bleeding component 58 for bleeding blood out of an examinee's skin. The bleeding component 58 can be a bleeding needle. The first stimulating component 54, the second stimulating component 56, and the bleeding component 58 are disposed at an end of the rod-type structure 52. A protruding end of the first stimulating component 54 or the second stimulating component 56 can be a blunt head. A protruding end of the bleeding component 58 can be a needle structure. A height of the bleeding component 58 is less than those of the first and second stimulating components 54, 56. A distance D1 between the first and second stimulating components 54, 56 is less than or equal to a two-point sense threshold of the human perception system. The bleeding component 56 is disposed between the first and second stimulating components 54, 56. The two-point sense threshold of the human perception system is a minimum distance between two stimulating points that the human perception system can distinguish these two stimulating points. That is, when a distance between two stimulating points is less than that of the two-point sense threshold of the human perception system, the human perception system regards these two stimulating points as one stimulating point. The two-point sense thresholds of the human perception system in different human parts are different. For example, the two-point sense threshold of the human perception system in a lower leg is 48 mm substantially, the two-point sense threshold in a palm is 13 mm substantially, the two-point sense threshold in a thumb is 3 mm substantially, and the two-point sense threshold in a forefinger is 2 mm substantially. The distance between the first and second stimulating components 54, 56 can be designed according to the human part that the bleeding apparatus 50 bleeds.

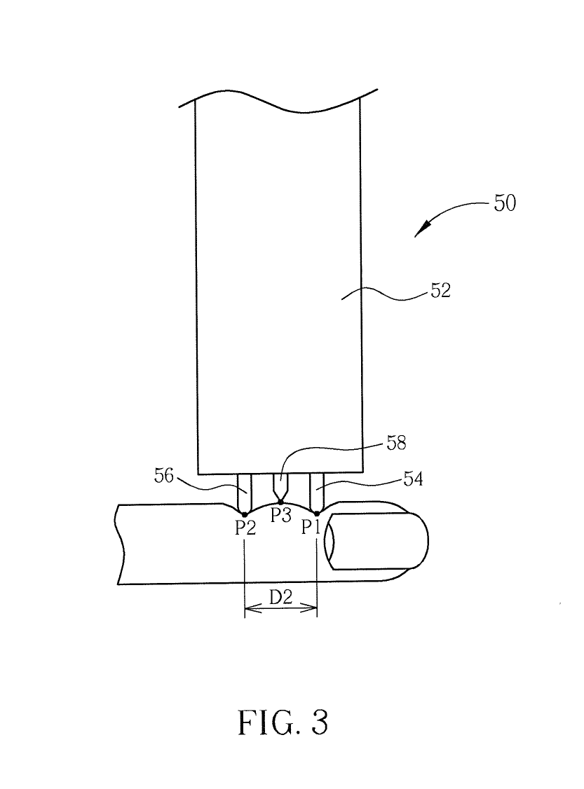

FIG. 3 illustrates a schematic diagram of the bleeding apparatus 50 of FIG. 2 bleeding the examinee. As shown in FIG. 3, the user can press the rod-type structure 52 to examinee's finger directly, and the first stimulating component 54 presses at a first simulating point P1 of the examinee's finger so as to induce sense perception of the examinee, and the second stimulating component 56 presses the examinee at a second simulating point P2 of the examinee's finger so as to induce sense perception of the examinee. The bleeding component 58 shorter than the first and second stimulating components 54, 56 can prick the examinee's skin for bleed with increasing pressure. The bleeding component 58 can be disposed inside the rod-type structure 52 in an extendable manner and be capable of protruding out of the rod-type structure 52 when turning on a switch (not shown in the drawings) on the rod-type structure 52 for bleeding the examinee. The bleeding component 58 also can be disposed at an end of the rod-type structure 52 directly for bleeding the examinee directly. In addition, the first and second stimulating components 54, 56 can be disposed on an attachment capable to be attached to the end of the rod-type structure 52. The first and second stimulating components 54, 56 also can be formed on the end of the rod-type structure 52 which contacts with skin directly. The distance D1 between the first and second stimulating components 54, 56 is less than or equal to the two-point sense threshold of the human perception system in the examinee's bleeding part. That is, a distance D2 between the first and second stimulating points P1, P2 is less than or equal to the two-point sense threshold of the human perception system in the examinee's bleeding part. The distance D1 and the distance D2 are substantially the same. When the first and second stimulating components 54, 56 press the examinee at the first and second simulating points P1, P2, respectively, to induce sense perception of the examinee, a bleeding point P3 of the bleeding component 58 between the first and second stimulating points P1, P2 is within the two-point sense threshold. The human perception system regards the first and second stimulating points P1, P2 as one stimulating point so that the human perception system can not sense the pain due to the bleeding component 58 pricking the examinee's skin so as to reduce the examinee's pain when bleeding the examinee.

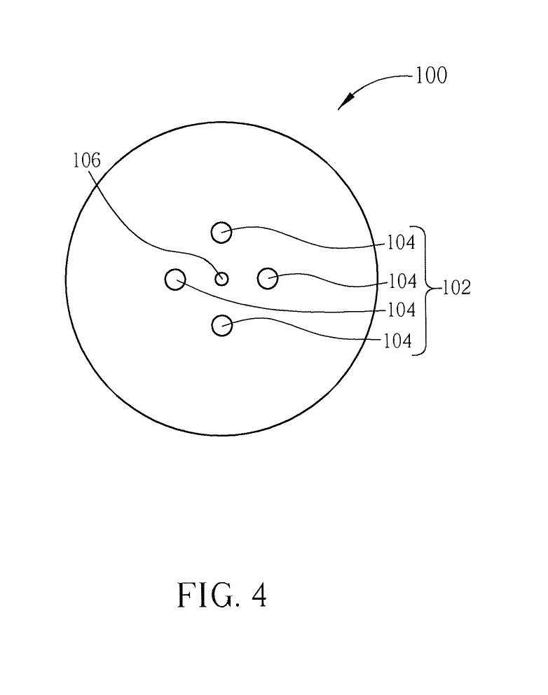

Furthermore, a stimulating component array can be formed by a plurality of stimulating components. Please refer to FIG. 4. FIG. 4 is an end view of a bleeding apparatus 100 according to a second embodiment of the present invention. The bleeding apparatus 100 includes a stimulating component array 102 having four stimulating components 104 in an annular arrangement. The distance between any two of the stimulating components 104 is less than or equal to the two-point sense threshold of the human perception system in the examinee's bleeding part. The bleeding apparatus 100 further includes a bleeding component 106 disposed in the center of the stimulating component array 102. The bleeding principle of reducing the examinee's pain of this embodiment is the same as mention above, and detailed description is hereby omitted.

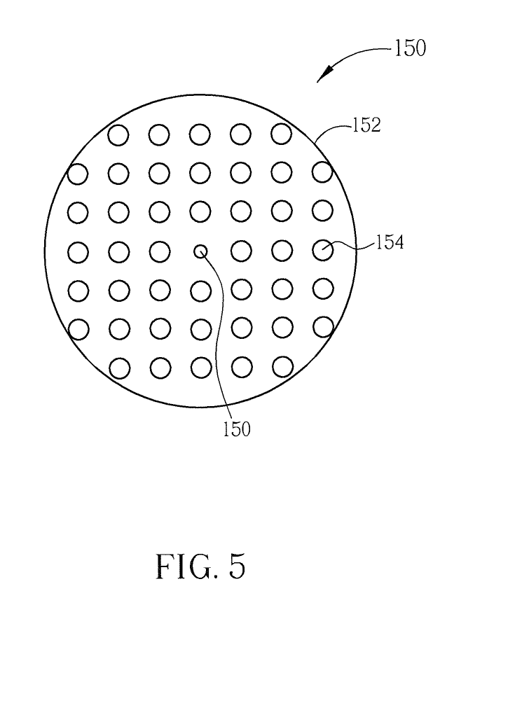

FIG. 5 is an end view of a bleeding apparatus 150 according to a third embodiment of the present invention. The bleeding apparatus 150 includes a stimulating component array 152 which is a circle with a diameter of 15 mm substantially. The stimulating component array 152 includes a plurality of stimulating components 154. The distance between the two adjacent stimulating components 154 is less than or equal to the two-point sense threshold of the human perception system in the examinee's bleeding part. The bleeding apparatus 150 further includes a bleeding component 156 disposed in the center of the stimulating component array 152. The bleeding principle of reducing the examinee's pain of this embodiment is the same as mention above, and detailed description is hereby omitted.

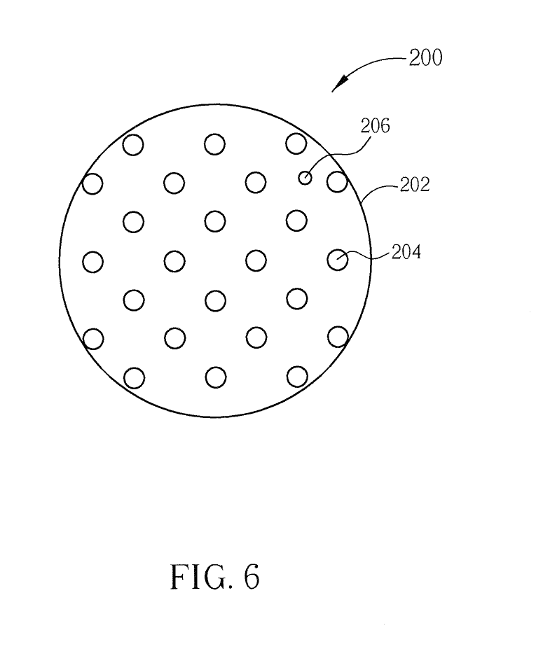

Please refer to FIG. 6. FIG. 6 is an end view of a bleeding apparatus 200 according to a fourth embodiment of the present invention. The bleeding apparatus 200 includes a stimulating component array 202 which is a circle type. A diameter of the circular stimulating component array 202 is 15 mm substantially. The stimulating component array 202 includes a plurality of stimulating components 204. The distance between any two adjacent stimulating components 204 is less than or equal to the two-point sense threshold of the human perception system in the examinee's bleeding part. The bleeding apparatus 200 further includes a bleeding component 206. The difference between the fourth embodiment and the third embodiment is that the bleeding component 206 is disposed on the rim of the stimulating component array 202 so as to reduce the examinee's pain efficiently when bleeding the examinee. The bleeding principle of reducing the examinee's pain of this embodiment is the same as mention above, and detailed description is hereby omitted.

In conclusion, the bleeding apparatus of the present invention stimulates the bleeding part with stimulating components wherein each distance between the stimulating components is less than or equal to the two-point sense threshold of the human perception system in the examinee's bleeding part. The human perception system regards stimulating points pressed by the stimulating components as one stimulating point so that the human perception system can not sense the pain due to the bleeding component stinging the examinee's skin for reducing the examinee's pain when bleeding the examinee. The structural and disposition design of the stimulating components is not limited to above-mentioned embodiments, and any mechanical design capable of making the human perception system regard stimulating points pressed by the stimulating components as one stimulating point is within the scope of the present invention.

In contrast to the prior art, the bleeding apparatus of the present invention stimulates the bleeding part with stimulating components wherein each distance between the stimulating components is less than or equal to the two-point sense threshold of the human perception system in the examinee's bleeding part. The human perception system regards stimulating points pressed by the stimulating components as one stimulating point so that the human perception system can not sense the pain due to the bleeding component stinging the examinee's skin so as to reduce the examinee's pain when bleeding the examinee.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A bleeding apparatus comprising:
a first stimulating component for pressing a first simulating point of an examinee;
a second stimulating component for pressing a second simulating point of the examinee; and
a bleeding component disposed between the first and second stimulating components for bleeding the examinee,
wherein a distance between the first and second simulating points is less than or equal to a two-point sense threshold of the examinee.

2. The bleeding apparatus of claim 1, wherein the bleeding component is a needle.

3. The bleeding apparatus of claim 1, wherein the distance between the first and second simulating points is depended on a human part where the bleeding apparatus bleeds.

4. The bleeding apparatus of claim 1, wherein an end of the first stimulating component or the second stimulating component for pressing is a blunt head.

5. The bleeding apparatus of claim 1, wherein a height of the bleeding component is less than those of the first and second stimulating components.

6. The bleeding apparatus of claim 1, wherein the first and second stimulating components constitute a stimulating component array.

7. The bleeding apparatus of claim 4, wherein the stimulating component array is circular.

8. The bleeding apparatus of claim 7, wherein the bleeding component is disposed in the center of the stimulating component array.

9. The bleeding apparatus of claim 7, wherein the bleeding component is disposed on the rim of the stimulating component array.

10. The bleeding apparatus of claim 7, wherein a diameter of the stimulating component array is 15 mm.

11. The bleeding apparatus of claim 1, further comprising a rod-type structure, and the first stimulating component, the second stimulating component, and the bleeding component are disposed at an end of the rod-type structure.

12. The bleeding apparatus of claim 11, wherein the bleeding component is disposed in a body in an extendable manner.

13. The bleeding apparatus of claim 12, wherein the bleeding component is capable of protruding out of the body when turning on a switch on the body.

14. The bleeding apparatus of claim 12, wherein the first and second stimulating components are disposed on an attachment capable to be attached to the end of the rod-type structure.

15. The bleeding apparatus of claim 1, wherein the two-point sense threshold is 2 mm.

16. The bleeding apparatus of claim 1, wherein the two-point sense threshold is 3 mm.

17. The bleeding apparatus of claim 1 wherein the two-point sense threshold is 13 mm.

18. The bleeding apparatus of claim 1 wherein the two-point sense threshold is 48 mm.
